(19) United States
(12) Patent Application Publication
(10) Pub. No.: US 2008/0300582 A1
(43) Pub. Date: Dec. 4, 2008

(54) LASER SKIN PERFORATOR AND METHOD OF FORMING SKIN HOLE USING LASER

(75) Inventor: Man-Su PARK, Daejeon-City (KR)

Correspondence Address: LEXYOUME IP GROUP, LLC, 5180 PARKSTONE DRIVE, SUITE 175, CHANTILLY, VA, 20151, US

(73) Assignee: DiNonA Inc., Seoul (KR)

(21) Appl. No.: 12/107,732

(22) Filed: Apr. 22, 2008

(30) Foreign Application Priority Data
Apr. 23, 2007 (KR) 10-2007-0039519

(51) Int. Cl. A61B 18/20

(52) U.S. Cl. 606/9

(57) ABSTRACT

A laser skin perforator and a method of forming a skin hole using a laser that forms a plurality of micro holes in skin using the laser when treating various skin diseases such as removing a wrinkle and a scar of skin are provided. The laser skin perforator includes a main laser oscillator, an optical unit, an area display laser oscillator, a shutter unit, and a laser emission unit. Further, the method of forming a skin hole using a laser includes setting a surgical operation area, radiating an area display laser beam, setting an emission value of a main laser beam, setting the pulse number of the main laser beam, and forming a micro hole in skin by radiating the main laser beam of the set pulse number.

### CROSS-REFERENCE TO RELATED APPLICATION

This application claims priority to and the benefit of Korean Patent Application No. 10-2007-0039519 filed in the Korean Intellectual Property Office on Apr. 23, 2007, the entire contents of which are incorporated herein by reference.

### BACKGROUND OF THE INVENTION

(a) Field of the Invention

The present invention relates to a laser skin perforator and a method of forming a skin hole using a laser. More particularly, the present invention relates to a laser skin perforator and a method of forming a micro hole in skin using a laser for the purpose of removing a skin wrinkle, removing a scar, and treating various skin diseases.

(b) Description of the Related Art

The word “LASER” is an abbreviation of “light amplified by stimulated emission of radiation,” and an operational principle thereof is to amplify light as one light particle induces the release of other particles in a medium of an excited state within a laser machine. The light particle induces the release of other light particles while again passing through the medium after being reflected by a mirror that is provided within the machine. Two mirrors exist within the machine, and because a mirror that is positioned at the front is transflective, the mirror passes some of the light particles therethrough. Light that is obtained in this way is parallel light that is bright and has a short wavelength, that temporarily or spatially has a coupling force, and that does not diffuse and is transmitted in parallel, and it is called a laser ray. That is, a laser is a device that converts an energy difference that is generated when returning an electron to an original position to light after raising it to a high energy level by applying external energy to the electron in any orbit, and that amplifies to have strong energy by gathering the light in one direction using a special device.

Lasers are categorized as a solid laser (a ruby laser, an alexandrite laser, etc.), a liquid laser (a pigment laser), a gas laser (carbon dioxide laser, etc.), and other semiconductor lasers according to the kind of a medium that is contained in a generation device thereof. The materials always generate light of a fixed wavelength according to their optical characteristics, and according to the wavelengths thereof, a place to use the laser is determined. Because the laser very strongly generates and radiates light of a fixed wavelength, the laser can be used for various purposes according to a wavelength and an energy level.

In a modern medicine, the laser has been used in various forms. A medical application of a laser that is conventionally limited to a surgical operation field has recently been widely extended to a non-invasive diagnosis and treatment field. The laser was first introduced for surgical operations, and the laser that was introduced for surgery had high output energy and performed a tissue destruction operation. The high output laser was used for tissue cutting, coagulation, and stanching through the tissue destruction operation. The laser that has been widely used in modern medicine is variously used for skin treatment and is used for the purpose of removing skin wrinkles and treating various skin diseases.

In general, there are various kinds of methods for removing a wrinkle. As a method using conventional plastic surgery, a method of removing and suturing flabby skin tissue is used, but the method has a problem in that a scar remains in the skin and the surgical operation cost is somewhat expensive. Further, when a skin wrinkle is light, a method of raising the skin by inserting a material, which deliquesces well with human body tissue, such as restylene, is used, but the method has a problem that rejection may occur when the material is absorbed into the skin tissue. Further, a method of grafting fat from the patient to a wrinkle portion may be used, but because the fat is absorbed into the tissue after a while, a long-term effect cannot be expected. As a method of eliminating movement of skin when making faces or laughing by paralyzing muscular motion of a wrinkle portion, a method of injecting a toxin called Botox into a wrinkle portion of a face may be used, but the method has a problem in that Botox should be repeatedly injected at an interval of 4-6 months and much cost is thus required.

In order to solve the problems, in a proposed surgical operation method that removes a wrinkle using a laser, when applying a hand piece that emits the laser to a surgical operation area of a patient, medical staff performed a surgical operation while moving the hand piece that emits the laser to the skin with a direct manual operation. However, in the conventional surgical operation method, there is a problem that emission density of a laser is not uniform due to the manual operation of the medical staff that operates on a patient.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a laser skin perforator and a method of forming a skin hole using a laser having advantages of automatically performing various skin surgical operation processes using a laser that is currently manually performed with a hand piece.

The present invention has been made in an effort to further provide a laser skin perforator and a method of forming a skin hole using a laser having advantages of performing a differentiated surgical operation according to a patient's state by directly setting treatment point density and energy level per pulse of the laser when removing a wrinkle and various scars of skin using the laser.

The present invention has been made in an effort to further provide a laser skin perforator and a method of forming a skin hole using a laser having advantages of adjusting a depth of a skin hole according to the number of emissions of the laser by emitting a plurality of laser pulses to any one position in forming a hole in skin using the laser.

The present invention has been made in an effort to further provide a laser skin perforator and a method of forming a skin hole using a laser having advantages of calling up and using a preset form of an outer boundary line for a surgical operation area of a patient.

An exemplary embodiment of the present invention provides a laser skin perforator including: i) a main laser oscillator that generates a main laser beam for forming a plurality of micro holes in a surgical operation area; ii) an optical unit that is connected to the main laser oscillator and that converts the main laser beam that is transferred from the main laser oscillator to parallel light; iii) an area display laser oscillator that generates an area display laser beam for radiating a laser beam to a surgical operation area that is set by a preset outer boundary line; iv) a shutter unit that is connected to the area display laser oscillator and the optical unit and that converts the main laser beam that is transferred from the optical unit and the area display laser beam that is transferred from the area display laser oscillator to either one according to a control situation; v) and a laser emission unit that directly radiates the area display laser beam and the main laser beam to the surgical operation area.

The laser skin perforator may further include a laser controller that is connected to the main laser oscillator and the area display laser oscillator and that controls the output of a laser beam; a central controller that is connected to the laser controller and the laser emission unit and that controls the laser controller and the laser emission unit; and an area display DB that is connected to the central controller, that stores various forms of preset outer boundary lines of the surgical operation area as data, and that calls up data of the stored outer boundary line when performing a surgical operation to provide the data to the central controller.

The central controller may include a control program that controls an oscillation of a laser and a position and operation of the laser emission unit, and the optical unit may include a parallel correction lens.

Another embodiment of the present invention provides a method of forming a skin hole using a laser, including: i) setting a surgical operation area in skin; ii) radiating an area display laser beam to a generated outer boundary line according to the set surgical operation area; iii) setting an emission value of a main laser beam to be radiated into the surgical operation area; iv) setting a pulse number of the main laser beam; and v) forming a micro hole of a predetermined depth in the skin by radiating a main laser beam of the set pulse number at a position point of the skin within the set surgical operation area.

The forming of a micro hole may include repeatedly forming micro holes while moving position within the set surgical operation area.

The setting of a surgical operation area may include setting a surgical operation area by drawing an outer boundary line of a surgical operation area through a touch screen, or calling up an outer boundary line form that is previously stored in an area display DB and setting a surgical operation area of skin along the called up outer boundary line form.

The forming of a micro hole may include forming a depth of a hole that forms in the skin in a range of 1 μm to 3000 μm.

A wavelength of the main laser beam may be in a range of 500 nm to 11,000 nm, and a spot size of the main laser beam may be in a range of 10 μm to 1000 μm.

The main laser beam may be formed as a pulse laser beam, a pulse width may be in a range of 500 μs to 1,000,000 μs, and an energy level per pulse may be in a range of 1 to 30 mJ.

A wavelength of the area display laser beam that is emitted after being generated through the area display laser oscillator may be in a range of 500 nm to 700 nm.

As described above, a laser skin perforator and a method of forming a skin hole using a laser of the present invention have the following effect.

First, in removing a wrinkle and treating a scar of a human body, a laser can be accurately and regularly radiated to a patient's surgical operation area, compared with the conventional art that manually radiates a laser to the patient's surgical operation area with a hand piece.

Second, because treatment point density and energy level per pulse of a main laser that is radiated to an affected part of the patient can be set, a differentiated surgical operation according to the patient's state can be performed.

Third, the patient's surgical operation area can be visually checked by an area display laser.

Fourth, a depth of a hole that is formed in skin can be adjusted by controlling the pulse number of a main laser beam.

Fifth, because all processes are automatically performed, the surgical operation can be performed quickly and accurately.

### BRIEF DESCRIPTION OF THE DRAWINGS

FIG. 1 is a schematic view illustrating a method of forming a skin hole using a laser according to an exemplary embodiment of the present invention.

FIG. 2 is a block diagram illustrating a configuration of a laser skin perforator according to an exemplary embodiment of the present invention.

FIG. 3 is a flowchart illustrating a method of forming a skin hole using a laser according to an exemplary embodiment of the present invention.

FIG. 4 is a perspective view illustrating a method of forming a skin hole using a laser according to an exemplary embodiment of the present invention.

FIG. 5 is a perspective view illustrating an external shape of a laser skin perforator using a laser according to an exemplary embodiment of the present invention.

FIG. 6 is a picture illustrating a microscopic treatment zone (MTZ) that is generated in guinea pig abdomen skin by a laser appliance according to an exemplary embodiment of the present invention.

FIG. 7 is a picture of an MTZ that is magnified to 400 times after H/E dyeing is executed in the MTZ of guinea pig abdomen skin that is generated by a laser appliance according to an exemplary embodiment of the present invention, where necrosis is observed in the epithelium and corium.

FIGS. 8A and 8B are pictures of an MTZ that is magnified to 100 times after H/E dyeing is executed in the MTZ of guinea pig abdomen skin that is generated by a laser appliance according to an exemplary embodiment of the present invention, where the MTZ was observed when 1 day and 3 days had elapsed after radiation.

FIGS. 9A to 9D are pictures of an MTZ that is magnified to 200 times after Van Gieson dyeing is executed in the MTZ of guinea pig abdomen skin that is generated by a laser appliance according to an exemplary embodiment of the present invention, where the MTZ was observed when 0 day, 1 day, 3 days, and 7 days had elapsed after radiation.

### DETAILED DESCRIPTION OF THE EMBODIMENTS

The present invention will be described more fully hereinafter with reference to the accompanying drawings, in which exemplary embodiments of the invention are shown. As those skilled in the art would realize, the described embodiments may be modified in various different ways, all without departing from the spirit or scope of the present invention. The drawings and description are to be regarded as illustrative in nature and not restrictive. Like reference numerals designate like elements throughout the specification.

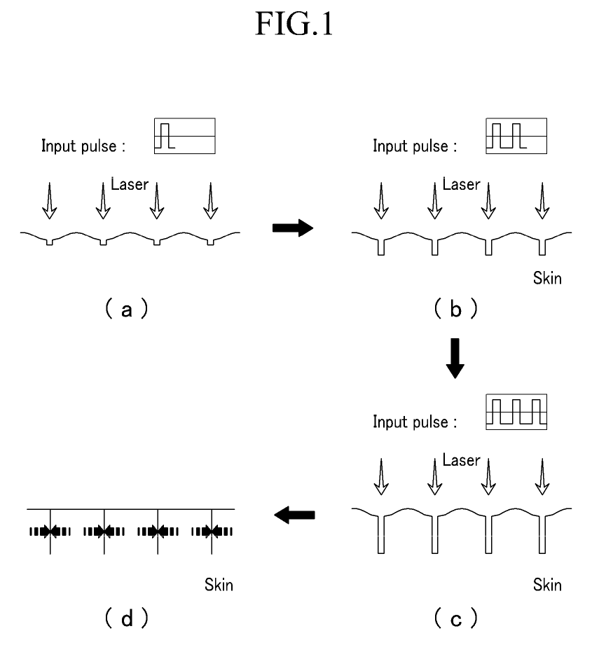

FIG. 1 is a schematic view illustrating a method of forming a skin hole using a laser according to an exemplary embodiment of the present invention.

As shown in FIG. 1, a laser is radiated to a wrinkled area of skin, as in FIG. 1(a). The depth of the formed hole can be adjusted by controlling the pulse number of a laser that is emitted with a multiple pulse, as in FIGS. 1(b) and 1(c). In this way, holes having a micro diameter and a predetermined depth meld together shortly after being generated, as shown in FIG. 1(d), and at this time, because the skin tissues pull each other, a wrinkle of the skin is unfolded.

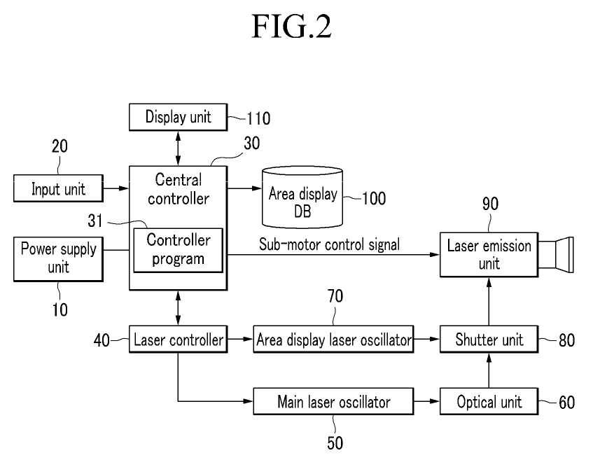

FIG. 2 is a block diagram illustrating a configuration of a laser skin perforator according to an exemplary embodiment of the present invention.

As shown in FIG. 2, the laser skin perforator according to the present exemplary embodiment includes a power supply unit 10, an input unit 20, a central controller 30, a laser controller 40, a main laser oscillator 50, an optical unit 60, an area display laser oscillator 70, a shutter unit 80, a laser emission unit 90, an area display DB 100, and a display unit 110.

The power supply unit 10 performs a function of supplying driving power to circuits and units.

The input unit 20 is a means that inputs a set value for radiating a laser beam to a patient's surgical operation area and can be a touch screen, for example, and when the touch screen is used as the input unit 20, the touch screen is used as both an input means and a display means.

An outer boundary line of a surgical operation range of a patient's skin to be radiated with a laser beam can be set through the input unit 20, and environment values such as treatment point density of a laser beam and an energy level per pulse can be set.

As a method of setting an outer boundary line of a surgical operation of a patient's skin, a method of drawing the outer boundary line of the surgical operation range of the patient's skin using an input means consisting of a touch screen and a method of calling up and using a specific shape of an outer boundary line that is previously set and stored from the area display DB 100 can be selectively used.

Treatment point density of a laser beam uses pulse per area (PPA) as a unit thereof and indicates the number of pulses per area of 1 cm². Further, the unit of energy level per pulse is represented by mJ, and the energy level is preferably set within a range of 1 to 30 mJ.

The central controller 30 applies an operation signal to the laser emission unit 90 and the laser controller 40 according to a value that is input by the input unit 20. Further, the central controller 30 performs an entire control function of the perforator, which receives a current status value and provides the output value to the display unit 110, etc.

The central controller 30 includes a control program 31 that controls an oscillation of a laser and a position and operation of the laser emission unit 90, and the control program 31 may be a program that is driven in a graphical user interface (GUI) environment (e.g. MS Windows).

The laser controller 40 controls general processes of a laser output including outputs of the main laser oscillator 50 and the area display laser oscillator 70, and performs functions of applying a control signal to each of the laser oscillators 50 and 70 to emit a laser beam according to a set value that a user inputs to the input unit 20, receiving a status signal for the output of the main laser oscillator 50, and providing the status signal to the central controller 30 so that the user may check the status signal in the display unit 110.

The main laser oscillator 50 performs a function of generating a laser beam for forming a plurality of micro holes in a surgical operation area of a patient's skin, and generates a laser beam according to an environment value that the user sets through the input unit 20, and the generated laser beam is transferred to the optical unit 60 and emitted.

Further, the main laser oscillator 50 emits a plurality of laser beam pulses by a multiple pulse in order to form a predetermined depth of hole at any position of surgical operation areas, and in order to implement this, oscillation of a laser is performed in a single mode.

A wavelength of a laser beam that is emitted after being generated through the main laser oscillator 50 is in a range of 500 nm to 11,000 nm. Further, the main laser oscillator 50 repeatedly emits a plurality of laser beam pulses, and when emitting a plurality of laser beam pulses, a pulse width of a laser beam is in a range of 500 μs to 1,000,000 μs.

The optical unit 60 is one of constituent elements that are required when outputting the generated laser beam, and it generates parallel light. The optical unit 60 can use a parallel correction lens, and a spot size of a position at which a laser beam that is output from the optical unit 60 reaches skin is in a range of 10 μm to 1000 μm.

When the user sets a surgical operation area through the input unit 20, the area display laser oscillator 70 performs a function of oscillating an area display laser so that an area display laser beam is radiated to a boundary line of the corresponding area.

The emitted area display laser beam is in a wavelength range of 500 nm to 700 nm, and preferably uses a wavelength of 635 nm.

The shutter unit 80 performs a function of converting a laser beam that is generated in the area display laser oscillator 70 and a laser beam that is generated in the main laser oscillator 50 to each other depending on to a control situation.

The laser emission unit 90 performs a function of directly radiating an area display laser and a main laser to a surgical operation area of a patient's skin. Because the laser emission unit 90 includes at least one sub-motor at the inside thereof, the laser emission unit 90 can quickly and accurately perform position control in an XY-shaft coordinate. The position control by the sub-motor includes a function of accurately expressing a boundary area of a surgical operation area that is set through the input unit 20 and radiating a laser within the corresponding range.

The area display DB 100 is a data storage means in which various forms of outer boundary lines of a surgical operation area are previously set and stored, and the user can directly draw a surgical operation area through the input unit 20 or call up and use various forms of outer boundary lines that are previously set in the area display DB 100.

The display unit 110 is a display means that displays a setting state for emitting a laser and a process of emitting a laser.

Hereinafter, a method of forming a skin hole using a laser according to an exemplary embodiment of the present invention will be described.

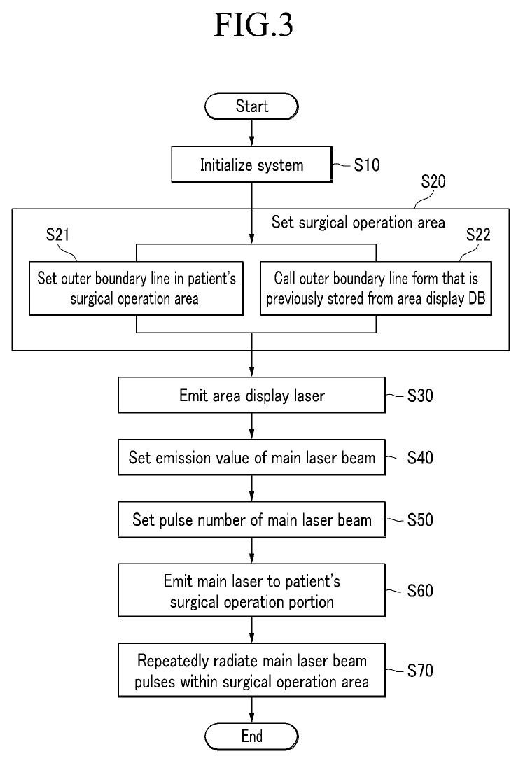

FIG. 3 is a flowchart illustrating a method of forming a skin hole using a laser according to an exemplary embodiment of the present invention.

First, after a system for a laser skin perforator is initialized (S10), a surgical operation area in a patient's skin is set (S20).

The step of setting a surgical operation area can be applied when selecting one of drawing an outer boundary line in a surgical operation area using a touch screen (S21) and calling up an outer boundary line form that is previously stored to the area display DB 100 and setting the surgical operation area (S22).

It is preferable that the surgical operation area is set to a circle with a diameter within 100 mm at step (S20) of setting a surgical operation area.

A laser that is generated by an operation of the area display laser oscillator 70 is emitted through the laser emission unit 90 to the outer boundary line that is set at step (S20) of setting a surgical operation area (S30), and the surgical operation area can be visually checked by radiating an area display laser beam to the skin surgical operation area. Further, the radiated area display laser beam can sustain the state until a surgical operation process is terminated.

Next, an emission value of a main laser beam that sets treatment point density and energy level per pulse of the main laser beam to be radiated into the set outer boundary line is set (S40), and then the pulse number of the main laser beam is set (S50).

Step (S50) of setting the pulse number of the main laser beam is to adjust a depth of a hole that is formed in the skin, and the depth of the hole that is formed in the skin can be adjusted by repeatedly radiating a plurality of laser beam pulses corresponding to the preset number of times at any position of the surgical operation area. The depth of the hole that is formed in the skin is in a range of 1 μm to 3000 μm.

Next, a predetermined depth of micro hole in skin is formed by radiating a plurality of main laser beam pulses the preset number of times at a position point of the skin within the set surgical operation area (S60), and then a plurality of main laser beam pulses are repeatedly radiated while moving position within the set surgical operation area (S70).

Further, the order of step (S20) of setting a surgical operation area, step (S30) of emitting an area display laser, step (S40) of setting an emission value of the main laser beam, and step (S50) of setting the pulse number of the main laser beam is not sequential, and even if the order of steps is changed, the order does not have an influence on a surgical operation effect.

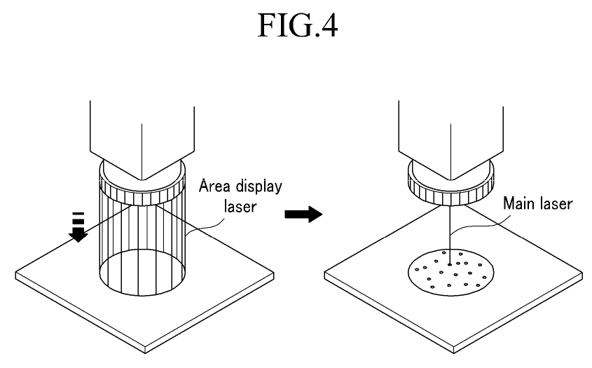

FIG. 4 is a perspective view illustrating a method of forming a skin hole using a laser according to an exemplary embodiment of the present invention. As shown in FIG. 4, after radiating an outer boundary line to a surgical operation area by an area display laser, the main laser forms a plurality of micro holes within the set outer boundary line. In this case, the main laser can form a plurality of micro holes while moving a position in a range that does not escape the outer boundary line.

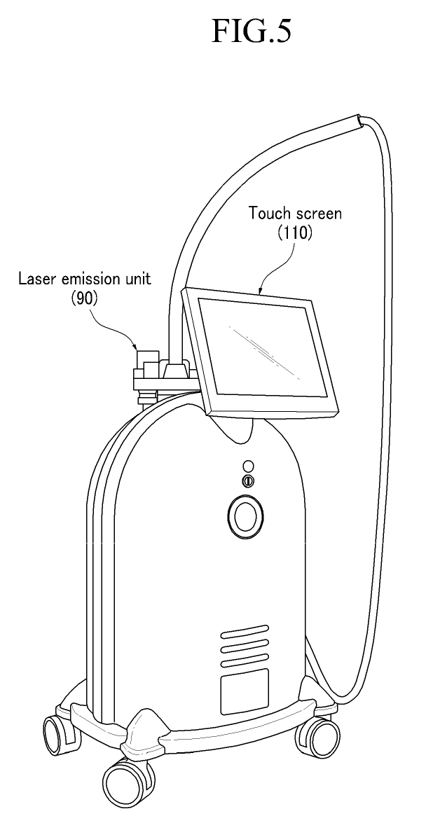

FIG. 5 is a perspective view illustrating an external shape of a laser skin perforator according to an exemplary embodiment of the present invention.

Experimental Example

After radiating guinea pig abdomen skin with a density of 784/cm² and a capacity of 10 mJ and 20 mJ using a laser medical appliance according to an exemplary embodiment of the present invention, in order to extract a radiation portion and perform a pathology histological test of a heat injury and a recovery process immediately, i.e. 0 days, and when 1 day, 3 days, and 7 days had elapsed after radiation, a differentiation light heating decomposition effect according to a radiation amount and a time passage was evaluated by performing hematoxylin & eosin (H/E) dyeing and Van Gieson dyeing.

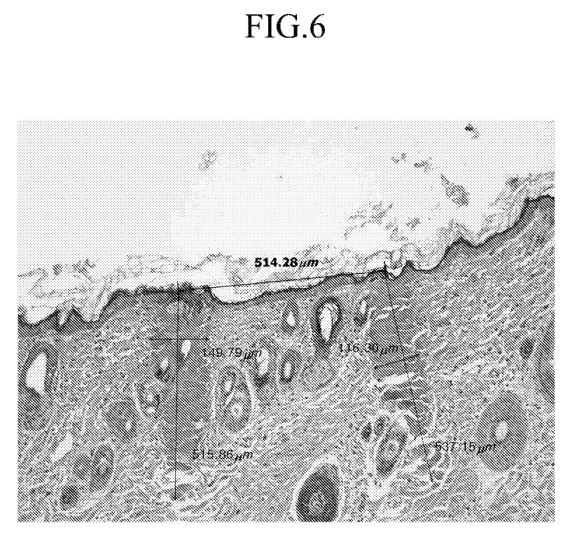

An MTZ was obviously observed with an interval of an average of 400 μm in the epithelium and corium that were injured by heat of all extracted specimens when 0 days had elapsed i.e., immediately after laser radiation. FIG. 6 representatively shows two clear cone shapes of MTZs having depths of 518.85 μm and 537.15 μm and widths of 149.79 μm and 116.30 μm. Average diameters of a microscopic laser beam were 105.61 μm and 130.57 μm when 10 mJ and 20 mJ, respectively, were radiated. Average transmission depths were 308.46 μm and 414.15 μm when 10 mJ and 20 mJ, respectively, were radiated.

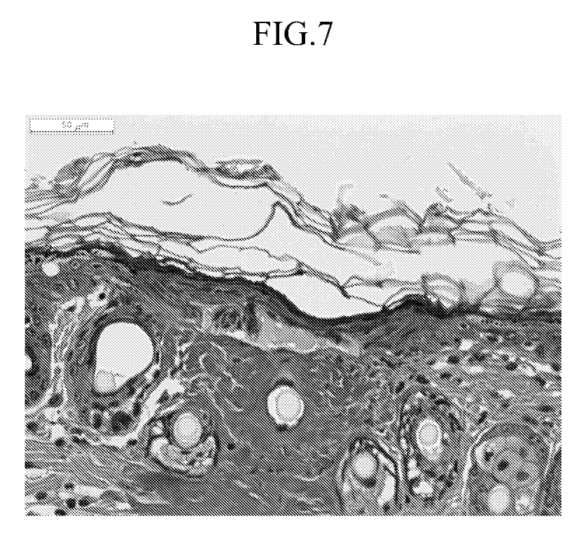

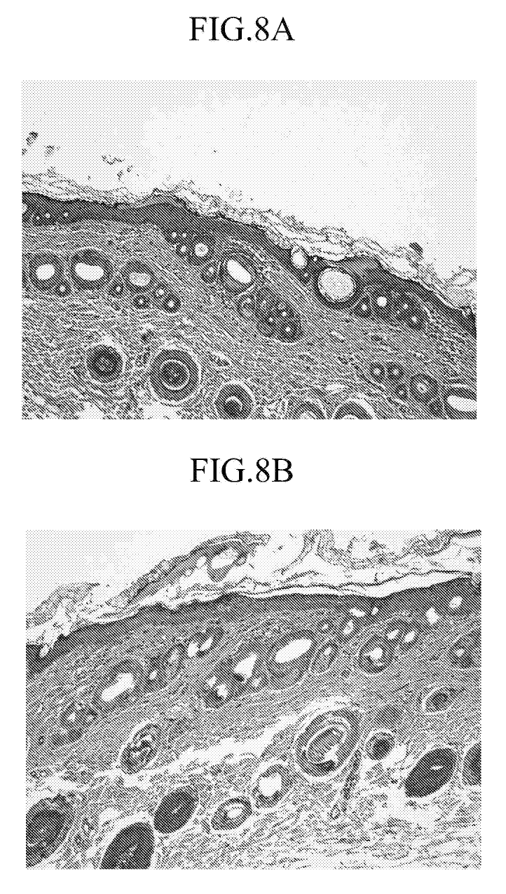

Referring to FIG. 7, necrosis of the epithelium tissue of a laser radiation area was observed and the stratum corneum was protected. Referring to FIGS. 8A and 8B, re-epithelium could be checked in all specimens when 1 day had elapsed after radiation. In the epithelium, when 1 day and 3 days had elapsed after radiation, multiple microscopic epithelial necrotic debris (MEND) portions were observed in an upper layer of a re-epithelium under the stratum corneum and were moved to the side of a heat injury portion, and finally eliminated from the skin. After 7 days had elapsed, the MEND was not observed in a specimen.

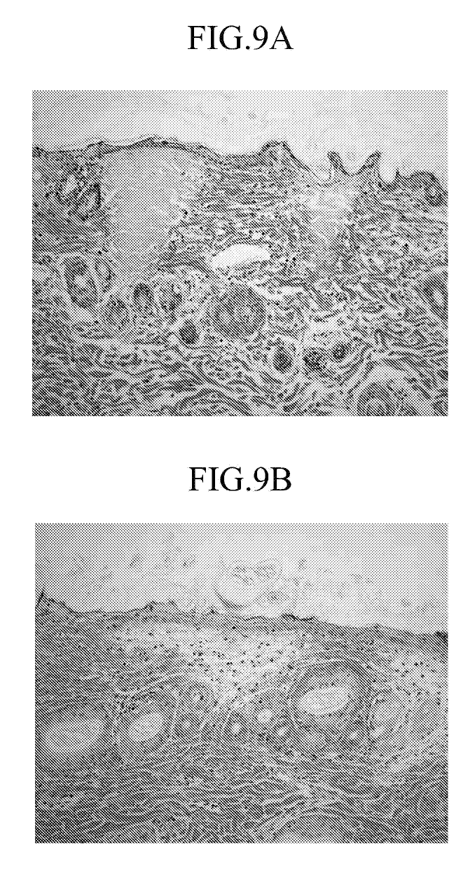

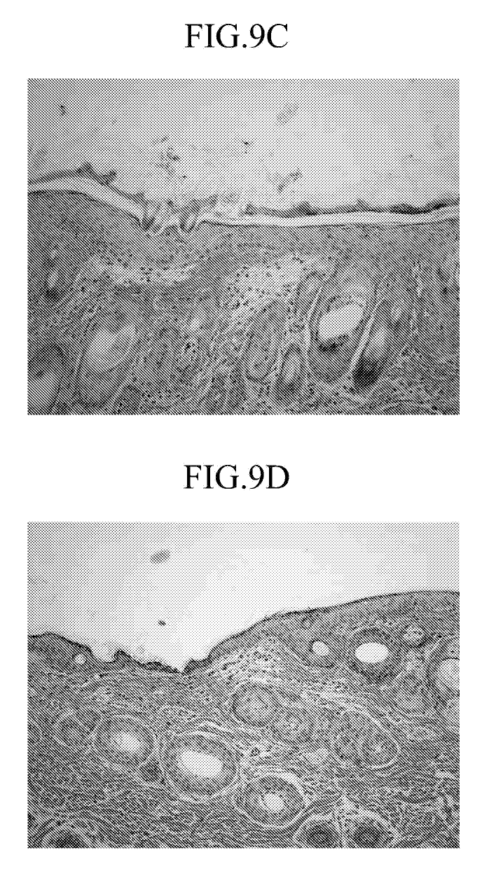

It was observed that an area of the MTZ within the corium was reduced by 30% when 1 day had elapsed after radiation, and it can be seen that a quick healing process thus occurred. Referring to FIGS. 9A to 9D, when 7 days had elapsed after radiation, collagen remodeling was completed in a guinea pig to a degree that it was hard to check the MTZ and thus it can be seen that it was normalized. In a process of injury and healing, an inflammation reaction or a granulation tissue was not observed.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A laser skin perforator comprising:
a main laser oscillator that generates a main laser beam for forming a plurality of micro holes in a surgical operation area;
an optical unit that is connected to the main laser oscillator and that converts a main laser beam that is transferred from the main laser oscillator to parallel light;
an area display laser oscillator that generates an area display laser beam for radiating a laser beam to a surgical operation area that is set by a preset outer boundary line;
a shutter unit that is connected to the area display laser oscillator and the optical unit and that converts the main laser beam that is transferred from the optical unit and the area display laser beam that is transferred from the area display laser oscillator to either one according to a control situation; and
a laser emission unit that directly radiates the area display laser beam and the main laser beam to the surgical operation area.

2. The laser skin perforator of claim 1, further comprising:
a laser controller that is connected to the main laser oscillator and the area display laser oscillator and that controls the output of the laser beam;
a central controller that is connected to the laser controller and the laser emission unit and that controls the laser controller and the laser emission unit; and
an area display DB that is connected to the central controller, and that stores various forms of preset outer boundary lines of the surgical operation area as data, and that calls up the stored outer boundary line data when performing a surgical operation to provide the data to the central controller.

3. The laser skin perforator of claim 2, wherein the central controller comprises a control program that controls an oscillation of a laser and a position and an operation of the laser emission unit.

4. The laser skin perforator of claim 1, wherein a wavelength of the main laser beam that is emitted after being generated through the main laser oscillator is in a range of 500 nm to 11,000 nm.

5. The laser skin perforator of claim 1, wherein the main laser beam that is emitted after being generated through the main laser oscillator is formed as a pulse laser beam, and a pulse width thereof is in a range of 500 μs to 1,000,000 μs.

6. The laser skin perforator of claim 1, wherein a spot size of the main laser beam that is emitted after being generated through the main laser oscillator is in a range of 10 μm to 1000 μm.

7. The laser skin perforator of claim 1, wherein the main laser beam that is emitted after being generated through the main laser oscillator is formed as a pulse laser beam, and an energy level per pulse thereof is in a range of 1 to 30 mJ.

8. The laser skin perforator of claim 1, wherein the optical unit comprises a parallel correction lens.

9. The laser skin perforator of claim 1, wherein a wavelength of an area display laser beam that is emitted after being generated through the area display laser oscillator is in a range of 500 nm to 700 nm.

10. A method of forming a skin hole using a laser, comprising:
setting a surgical operation area in skin;
radiating an area display laser beam to a generated outer boundary line according to the set surgical operation area;
setting an emission value of a main laser beam to be radiated into the surgical operation area;
setting the pulse number of the main laser beam; and
forming a micro hole of a predetermined depth in the skin by radiating a main laser beam of the set pulse number to a position point of the skin within the set surgical operation area.

11. The method of claim 10, wherein the forming of a micro hole comprises repeatedly forming micro holes while moving position within the set surgical operation area.

12. The method of claim 10, wherein the setting of a surgical operation area comprises drawing an outer boundary line of the surgical operation area through a touch screen.

13. The method of claim 10, wherein the setting of a surgical operation area further comprises calling up an outer boundary line form that is previously stored in an area display DB and setting a surgical operation area of skin along the called up outer boundary line form.

14. The method of claim 10, wherein a wavelength of the main laser beam is in a range of 500 nm to 11,000 nm.

15. The method of claim 10, wherein the main laser beam is formed as a pulse laser beam, and a pulse width thereof is in a range of 500 μs to 1,000,000 μs.

16. The method of claim 10, wherein a spot size of the main laser beam is in a range of 10 μm to 1000 μm.

17. The method of claim 10, wherein the main laser beam is formed as a pulse laser beam, and an energy level per pulse thereof is in a range of 1 to 30 mJ.

18. The method of claim 10, wherein a wavelength of the area display laser beam is in a range of 500 nm to 700 nm.

19. The method of claim 10, wherein the forming of a micro hole comprises forming a depth of a hole that forms in the skin in a range of 1 μm to 3000 μm.
